(19) United States
(12) Patent Application Publication
(10) Pub. No.: US 2026/0242810 A1
(43) Pub. Date: Aug. 20, 2026

(54) Virus-Mediated Systemic Genome Editing in Plants Without Regeneration

(71) Applicant: BETTERSEEDS LTD, Givat Chen (IL)

(72) Inventors: Tal SHERMAN, Rechovot (IL); Ido MARGALIT, Gan Yavne (IL); Shira COREM, Sdei Hemed (IL)

(73) Assignee: BETTERSEEDS LTD, Givat Chen (IL)

(21) Appl. No.: 19/453,263

(22) Filed: Jan. 20, 2026

Related U.S. Application Data
(60) Provisional application No. 63/747,378, filed on Jan. 21, 2025.

(51) Int. Cl. C12N 15/82; C12N 9/22; C12N 15/11

(52) U.S. Cl. CPC C12N 15/8213; C12N 9/226; C12N 15/11; C12N 15/8203; C12N 2310/20

(57) ABSTRACT

Systems and methods are provided for virus-mediated genome editing in plants that enable systemic, whole-plant editing and generation of stable, heritable genetic modifications without tissue culture or regeneration. Plant viral vectors are used to deliver genome editing components throughout intact plants. Viral cargo constraints are addressed by employing compact programmable nucleases or split nuclease configurations that are reconstituted within plant cells. Systemic viral movement enables genome editing activity in meristematic and reproductive tissues, allowing recovery of edited progeny directly from infected plants. The disclosed systems expand the applicability of genome editing to elite plant germplasm and enable regeneration-free production of heritable genome modifications.

### CROSS-REFERENCE TO RELATED APPLICATIONS

This application claims the benefit of priority under 35 U.S.C. § 119(e) to U.S. Provisional Patent Application No. 63/747,378, filed on Jan. 21, 2025, entitled “Manipulating Plant Gene Expression by Using Viral Vectors”, the entire contents of which are incorporated herein by reference.

### STATEMENT REGARDING FEDERALLY SPONSORED RESEARCH

This invention was not made with government support, and no governmental rights are associated with the invention.

### INCORPORATION BY REFERENCE

All publications, patents, and patent applications cited herein are incorporated by reference in their entirety for all purposes to the extent they are not inconsistent with the present disclosure.

### BACKGROUND OF THE INVENTION

### Plant Genetic Transformation Technologies

Plant genetic transformation has been a foundational tool in plant biology and crop improvement, enabling the introduction, modification, or suppression of genes to study function and improve agronomic traits.

Traditional transformation approaches rely primarily on Agrobacterium tumefaciens-mediated DNA transfer or physical delivery methods such as particle bombardment. While these technologies have enabled the commercialization of numerous transgenic crops, they remain technically demanding and limited in scope.

In most plant species, transformation protocols require delivery of foreign DNA into somatic plant cells followed by selection and regeneration of whole plants through tissue culture. These steps are often highly genotype dependent and require extensive optimization for each species and cultivar. Many economically important crops remain difficult or impractical to transform using conventional methods, particularly elite commercial varieties that have not been adapted to tissue culture conditions.

Furthermore, tissue culture and regeneration procedures can introduce unintended genetic and epigenetic changes, including somaclonal variation, chromosomal rearrangements, and altered gene expression patterns. These effects complicate downstream breeding and phenotypic evaluation and can reduce the predictability of trait performance. As a result, transformation efficiency, regeneration capacity, and genotype specificity remain major barriers to widespread deployment of advanced genetic technologies in plants.

### Genome Editing in Plant Science

Genome editing technologies have transformed the ability to generate targeted genetic modifications in plants. Site-specific endonucleases such as zinc-finger nucleases (ZFNs), transcription activator-like effector nucleases (TALENs), and CRISPR-associated nucleases enable precise modification of endogenous genomic loci through the introduction of double-strand breaks or targeted base changes.

Among these systems, CRISPR-based genome editing has been widely adopted due to its simplicity, programmability, and efficiency. In CRISPR systems, a guide RNA molecule directs a nuclease to a complementary DNA sequence, enabling targeted cleavage and subsequent DNA repair by endogenous cellular mechanisms. These repair processes can generate insertions, deletions, substitutions, or other sequence alterations.

Despite the conceptual simplicity of genome editing, practical implementation in plants remains constrained by delivery challenges. Genome editing reagents must still be introduced into plant cells, expressed at sufficient levels, and transmitted through cell divisions to generate stable and heritable modifications. In most cases, delivery of genome editing components relies on the same transformation and regeneration pipelines used for transgenesis, thereby inheriting the same technical limitations.

### Challenges in Plant Transformation and Regeneration

A major challenge in plant genome editing is the reliance on tissue culture-based regeneration systems. Many plant species exhibit low regeneration efficiency, and regeneration protocols often work only for a limited set of laboratory genotypes. Elite cultivars frequently display poor responsiveness to tissue culture, limiting the direct application of genome editing technologies in commercially relevant germplasm.

Even when regeneration is successful, the process can take several months and requires specialized infrastructure and expertise. The need to regenerate plants from transformed cells significantly slows the development cycle and limits scalability. These constraints are particularly problematic for applications requiring rapid iteration across multiple target genes or alleles.

Alternative delivery approaches, such as delivery of ribonucleoprotein complexes or transient expression systems, have been explored to reduce stable DNA integration. However, these approaches often still require regeneration from transformed tissues or protoplasts and are limited to a small subset of plant species. Consequently, transformation and regeneration remain the dominant bottlenecks in plant genome engineering.

### Viral Vectors as Delivery Tools in Plants

Plant viruses have been widely used as tools for transient gene expression, virus-induced gene silencing, and functional genomics. Viral vectors replicate autonomously, achieve high expression levels, and often move systemically throughout infected plants. These properties make viral vectors attractive candidates for delivering functional molecules into plants without stable genomic integration.

Viral delivery systems can introduce genetic material into differentiated plant tissues without the need for tissue culture. Because viral genomes replicate episomally and do not integrate into the host genome, viral vectors can provide transient expression while minimizing permanent foreign DNA insertion. In addition, systemic viral movement enables distribution of expressed molecules beyond the initial infection site.

As a result, viral vectors have been proposed as a means to bypass traditional transformation bottlenecks and simplify delivery of genome editing reagents in plants.

### Challenges of Viral Delivery for Genome Editing

Despite their advantages, viral vectors present significant challenges for genome editing applications. Many plant viruses have strict cargo size limitations, which restrict the length of foreign sequences that can be stably maintained within the viral genome. Large programmable nucleases, such as Cas9 and certain Cas12 variants, often exceed these limits, leading to instability or loss of inserted sequences during viral replication.

As a result of these cargo size constraints, prior virus-based genome editing approaches have frequently relied on regeneration of infected tissues to recover edited plants, as systemic movement and stable expression of large nucleases could not be maintained throughout the intact plant. This reliance on regeneration undermines the principal advantage of viral delivery and reintroduces the technical and biological limitations associated with tissue culture-based methods.

In addition, viral genomes can undergo deletion, recombination, or rearrangement when foreign sequences impose selective pressure on replication efficiency. These events can reduce consistency of nuclease expression and limit reproducibility of editing outcomes. Moreover, not all viral platforms efficiently access meristematic or reproductive tissues, which are critical for generating heritable genome modifications.

Some virus-based genome editing approaches have therefore relied on regeneration of infected tissues to recover edited plants, reintroducing the very tissue culture bottlenecks that viral delivery seeks to avoid. As a result, prior viral genome editing strategies have not fully realized the potential of virus-mediated delivery for heritable, whole-plant genome modification.

### Need for Improved Virus-Based Genome Editing Systems

Accordingly, there remains a need for genome editing systems that combine the systemic delivery advantages of viral vectors with strategies that overcome cargo size limitations and enable access to tissues responsible for heritable transmission. In particular, there is a need for approaches that allow genome editing components to be delivered throughout intact plants and produce stable, heritable modifications without reliance on tissue culture or regeneration.

The present invention addresses these needs by providing virus-based genome editing systems that employ nuclease configurations compatible with viral delivery constraints and enable systemic and heritable genome modification in plants.

### SUMMARY OF THE INVENTION

The present invention provides systems and methods for virus-mediated genome editing in plants that enable systemic, whole-plant genome editing and generation of stable, heritable genetic modifications without tissue culture or regeneration.

The invention is based on the use of nuclease architectures that are compatible with viral cargo and stability constraints, including compact programmable nucleases and split nuclease configurations that are reconstituted within plant cells. Systemic viral delivery enables genome editing activity in meristematic and reproductive tissues, allowing recovery of edited progeny directly from infected plants.

In these embodiments, access to tissues that contribute to germline development is an intended design outcome, such that viral delivery results in heritable genome modifications without requiring regeneration or selection of edited cells.

### BRIEF DESCRIPTION OF THE DRAWINGS

Exemplary, non-limiting embodiments of the disclosed subject matter are described with reference to the accompanying figures. The figures are not necessarily drawn to scale, and any dimensions shown are provided for illustrative purposes only and are not intended to be limiting. Corresponding or like elements may be designated by the same reference numerals or letters.

FIG. 1 is a schematic description of using intein moieties for split Cas9 reconstitution.

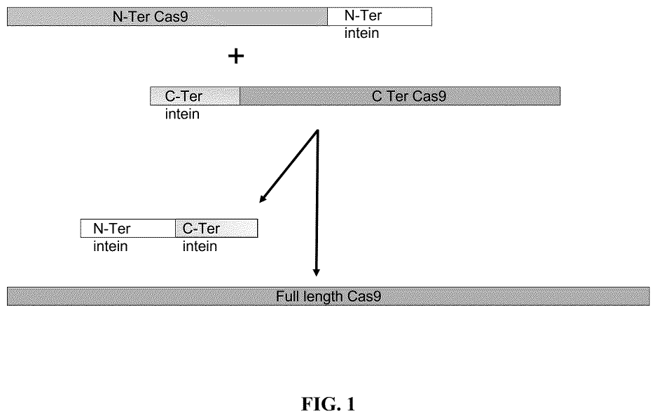

FIG. 2 is a graphical representation showing genome editing efficiency measured by indel percentage (%).

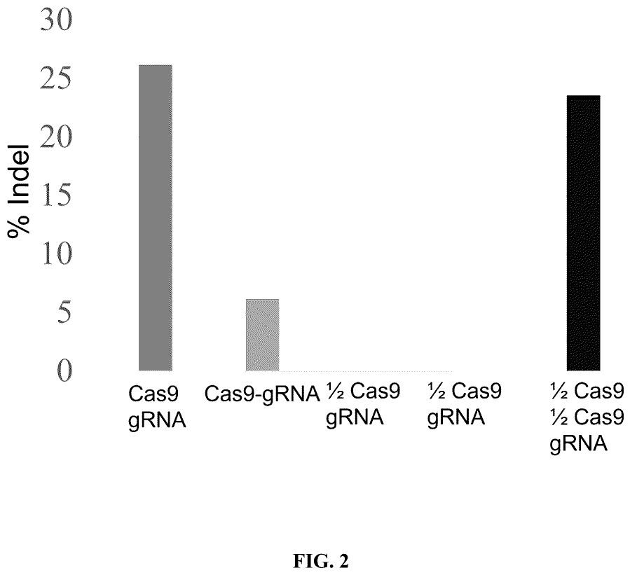

FIG. 3 is a photographic representation of a leaf infiltrated with an intact Cas9 expression cassette (FIG. 3A) or with a split Cas9 setup (FIG. 3B).

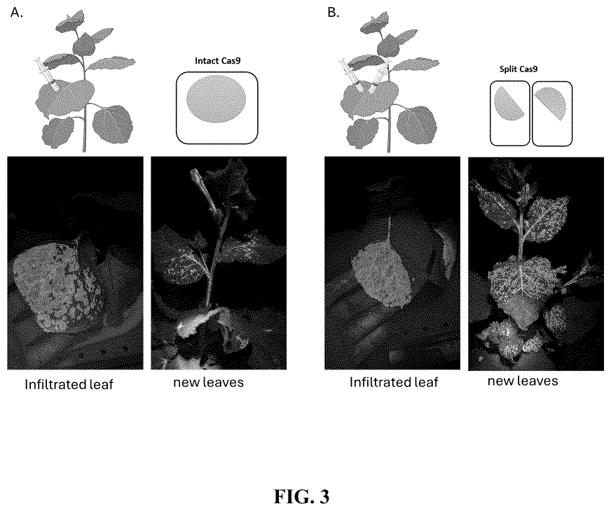

### DETAILED DESCRIPTION OF THE INVENTION

### Definitions and General Principles

Unless otherwise defined, all technical and scientific terms used herein have the same meaning as commonly understood by one of ordinary skill in the art of plant molecular biology and genome engineering.

As used herein, the term “virus-based vector” refers to a plant virus or viral replicon capable of autonomous replication and expression of encoded sequences within a plant host. Virus-based vectors may comprise DNA or RNA genomes and may be derived from naturally occurring plant viruses that are engineered to carry heterologous sequences.

The term “systemic movement” refers to the ability of a viral vector to move beyond the initial site of inoculation and spread to distal tissues of the plant, including newly developing leaves, meristematic tissues, and reproductive organs.

The term “heritable genome modification” refers to a genetic alteration introduced into a plant genome that is transmitted to progeny plants through sexual or asexual reproduction and is detectable in subsequent generations.

A “site-specific endonuclease” refers to an enzyme capable of introducing a targeted DNA modification at a defined genomic locus. Such enzymes include, without limitation, CRISPR-associated nucleases, transcription activator-like effector nucleases, zinc-finger nucleases, and meganucleases.

A “split endonuclease” refers to a site-specific endonuclease that is expressed as two or more separate polypeptide fragments that are individually inactive but capable of assembling or being reconstituted into a functional enzyme within a plant cell.

### Overview of Virus-Induced Genome Editing

The present invention provides systems and methods for virus-induced genome editing in plants, in which viral vectors are used to deliver genome editing components directly into intact plants. Viral replication enables high-level expression of editing reagents without stable integration of foreign DNA into the plant genome.

In contrast to conventional transformation-based approaches, the disclosed systems do not require regeneration of plants from transformed cells. Instead, genome editing activity is achieved in planta through systemic distribution of viral vectors, enabling editing events to occur in a broad range of tissues.

Systemic delivery allows genome editing components to reach tissues that contribute to reproductive development, thereby enabling the recovery of heritable genome modifications in progeny plants and seeds.

Accordingly, the systems described herein are architected around viral cargo constraints and systemic movement requirements. Selection of nuclease size, configuration, and delivery strategy is driven by the need to maintain viral stability and whole-plant infectivity while achieving genome editing activity in tissues capable of contributing to heritable transmission.

### Viral Vector Platforms

Viral vectors suitable for use in the invention are derived from plant viruses capable of systemic movement within the plant host. Such viruses may comprise DNA or RNA genomes and may be engineered to express genome editing components under control of viral or heterologous regulatory elements.

In various embodiments, viral vectors are derived from plant viruses belonging to families selected from Geminiviridae, Virgaviridae, Tobamoviridae, Potyviridae, Potexviridae, and Tospoviridae. These viral families include representatives known to replicate efficiently in plants and to spread beyond the site of inoculation.

Viral genomes may be modified to include coding sequences for genome editing components, guide RNAs, regulatory elements, or combinations thereof. Inserted sequences may be expressed from subgenomic promoters, duplicated regulatory regions, or other expression strategies compatible with viral replication and movement.

In certain embodiments, multiple viral vectors may be used concurrently to deliver different components of a genome editing system to the same plant.

### Modes of Viral Delivery

Virus-based vectors may be introduced into plants using any method suitable for initiating infection. Representative delivery methods include Agrobacterium-mediated delivery of viral constructs, mechanical inoculation, infiltration, or other techniques known in the art.

Delivery may be performed on seedlings, juvenile plants, or mature plants. In some embodiments, delivery is performed on vegetative tissues such as leaves or stems, from which the virus spreads systemically throughout the plant.

Importantly, the disclosed methods do not require regeneration of plants from infected tissues, thereby avoiding the need for tissue culture or selection steps.

### Programmable Nuclease Systems

The invention employs programmable nuclease systems to introduce targeted genome modifications. Suitable nucleases include CRISPR-associated nucleases, transcription activator-like effector nucleases, zinc-finger nucleases, and meganucleases.

In preferred embodiments, CRISPR-associated nucleases are used due to their programmability and efficiency. CRISPR nucleases may be directed to a target genomic locus by a guide RNA comprising a sequence complementary to the target site.

Genome editing may result in insertions, deletions, substitutions, or combinations thereof at the target locus, depending on the repair pathway engaged following nuclease activity.

### Compact Programmable Nucleases

In certain embodiments, the invention employs compact programmable nucleases that are smaller in size than commonly used nucleases such as Streptococcus pyogenes Cas9. Compact nucleases may include Cas12f, Cas121, CasΦ, and variants thereof.

The reduced coding sequence length of compact nucleases facilitates their incorporation into viral vectors with limited cargo capacity. Compact nucleases may be expressed directly from a single viral genome without compromising viral stability or systemic movement.

### Split Endonuclease Systems

In other embodiments, large nucleases are expressed as split endonuclease systems. In such systems, a nuclease is divided into two or more fragments, each encoded separately and delivered to plant cells.

The fragments may be fused to complementary intein sequences that mediate protein splicing. Upon co-expression within the same plant cell, the intein fragments catalyze reconstitution of the full-length functional nuclease.

Split endonuclease systems enable delivery of large genome editing enzymes using viral vectors that would otherwise be unable to accommodate the full-length coding sequence.

In this context, split nuclease architectures are not optional alternatives but enabling configurations that permit systemic viral delivery and germline-accessible genome editing where intact nuclease delivery is not feasible.

### Guide RNA Expression and Delivery

Guide RNAs may be expressed from viral genomes or from separate expression constructs delivered to the plant. In some embodiments, guide RNAs are encoded within the same viral vector as the nuclease. In other embodiments, guide RNAs are delivered from a second viral vector that co-infects the plant.

In embodiments where the plant constitutively expresses a nuclease, viral vectors may be used to deliver only guide RNAs, thereby conferring genome editing activity without introducing nuclease coding sequences.

### Systemic Editing and Access to Reproductive Tissues

Systemic movement of viral vectors enables genome editing components to reach tissues beyond the initial site of infection. In particular, viral vectors may access meristematic regions and tissues that contribute to reproductive development.

Editing events occurring in these tissues can give rise to heritable genome modifications that are transmitted to progeny plants and seeds. As a result, edited plants may be recovered directly from infected plants without regeneration.

### Genome Editing Outcomes and Heritability

Genome modifications introduced using the disclosed methods may include insertions, deletions, substitutions, or combinations thereof. Editing outcomes may be confirmed by molecular analysis of target loci in plant tissues or progeny plants.

Heritable genome modifications may be detected in seeds or progeny plants derived from infected individuals, demonstrating stable transmission of the edited genotype.

### Advantages Over Conventional Transformation

The virus-based genome editing systems disclosed herein provide several advantages over conventional transformation-based approaches.

These advantages include reduced reliance on tissue culture and regeneration, applicability to a broader range of plant genotypes, and the ability to generate edited plants more rapidly.

By leveraging systemic viral delivery and nuclease configurations compatible with viral vectors, the disclosed methods enable efficient, whole-plant genome editing and recovery of heritable modifications.

### EXAMPLES

The following examples are provided to illustrate representative embodiments of the invention. These examples are not intended to limit the scope of the invention, which is defined by the claims.

### Example 1

### Virus-Based Delivery of Split Site-Specific Endonuclease Components Construct Design and Assembly

Genome editing constructs were designed to enable virus-mediated delivery of a split site-specific endonuclease system. A programmable nuclease was divided into two polypeptide fragments corresponding to an N-terminal portion and a C-terminal portion of the enzyme. Each fragment was fused to a complementary intein sequence capable of mediating protein splicing upon co-expression in the same cell.

Coding sequences for the split nuclease fragments were inserted into plant virus-derived vector backbones under the control of viral regulatory elements. In certain embodiments, the nuclease fragments were expressed from separate viral vectors capable of co-infecting the same plant host. Guide RNA expression cassettes were included either within the same viral genome or within a second compatible viral vector.

All constructs were verified by restriction digest and sequence analysis prior to use.

### Plant Material and Growth Conditions

Plants used for viral delivery experiments were grown under standard greenhouse or growth chamber conditions suitable for the host species.

Plants were maintained under controlled light, temperature, and humidity conditions to support viral infection and systemic spread.

Plants were selected at a developmental stage suitable for viral inoculation, typically during vegetative growth when leaves were fully expanded and amenable to infection.

### Viral Inoculation and Delivery

Virus-based vectors were introduced into plants using Agrobacterium-mediated delivery of viral constructs. Agrobacterium cultures harboring the viral constructs were prepared using standard molecular biology techniques and adjusted to appropriate optical density prior to infiltration.

Infiltration was performed on leaf tissues using syringe infiltration or comparable methods. In certain embodiments, multiple viral constructs were co-delivered to enable expression of different components of the split nuclease system within the same plant.

Following infiltration, plants were maintained under growth conditions conducive to viral replication and systemic movement. Viral spread was monitored by visual inspection and, where applicable, reporter gene expression.

### Genome Editing Analysis

Genome editing activity was assessed by molecular analysis of target genomic loci. Tissue samples were collected from leaves distal to the initial inoculation site, indicating systemic delivery of the viral vectors.

Genomic DNA was extracted from plant tissues using standard extraction protocols. Target loci were amplified by polymerase chain reaction using primers flanking the expected nuclease cleavage site. Amplified products were analyzed by sequencing or other suitable methods to detect insertions, deletions, or other sequence modifications.

Editing efficiency was quantified by calculating the proportion of modified alleles relative to total alleles analyzed.

### Observations

Editing events consistent with site-specific nuclease activity were detected in systemically infected tissues. The presence of insertions and deletions at the target locus demonstrated functional reconstitution of the split nuclease in planta following viral delivery.

These results illustrate that virus-based delivery of split nuclease components can achieve genome editing activity without regeneration of transformed cells.

### Example 2

### Systemic Genome Editing and Reporter Gene Restoration Reporter System Design

To evaluate functional genome editing outcomes, a reporter gene system was used in which a reporter gene contained a disruption at a defined genomic locus. Successful genome editing at the target site was expected to restore reporter gene function, providing a visual or molecular readout of editing activity.

Plants harboring the disrupted reporter gene were used as hosts for viral delivery of genome editing components.

### Viral Delivery of Editing Components

Virus-based vectors encoding split nuclease fragments and guide RNAs targeting the disrupted reporter locus were introduced into plants as described in Example 1. In certain embodiments, intact nuclease constructs were also delivered for comparison.

Following inoculation, plants were maintained under conditions that allowed systemic spread of the viral vectors.

### Assessment of Reporter Gene Activity

Reporter gene activity was monitored in newly developed tissues distal to the inoculation site. Restoration of reporter gene expression was assessed by visual inspection, fluorescence imaging, or other suitable detection methods depending on the reporter system used.

Restored reporter activity indicated successful genome editing at the target locus and functional correction of the disrupted gene.

### Molecular Confirmation of Editing

Genomic DNA was extracted from tissues exhibiting restored reporter activity. Target loci were amplified and analyzed to confirm sequence modifications consistent with site-specific nuclease activity.

The presence of sequence changes at the expected genomic position confirmed that reporter restoration resulted from genome editing rather than transient expression.

### Observations

Reporter gene restoration was observed in systemically infected tissues, demonstrating that virus-based delivery of genome editing components can result in functional genome modification at the whole-plant level. Split nuclease systems exhibited robust activity and, in certain embodiments, improved systemic performance relative to intact nuclease constructs.

### Example 3

### Recovery of Heritable Genome Modifications Seed Collection and Progeny Analysis

Plants infected with viral vectors carrying genome editing components were allowed to develop normally and produce seeds. Seeds were collected and germinated under standard conditions to generate progeny plants.

Progeny plants were analyzed for the presence of genome modifications at the target locus.

### Genotypic Analysis of Progeny

Genomic DNA was extracted from progeny plants. Target loci were amplified and analyzed to detect the presence of sequence modifications introduced by genome editing.

Edited alleles detected in progeny plants demonstrated that genome editing events introduced by virus-based delivery were heritable and transmitted through seed.

### Observations

Heritable genome modifications were detected in progeny plants derived from virus-infected individuals. These results demonstrate that virus-based genome editing can produce stable, heritable genetic modifications without tissue culture or regeneration.

### Example 4

Comparison with Conventional Transformation-Based Editing Comparative Framework

To contextualize virus-based genome editing, the performance of viral delivery systems was compared conceptually with conventional transformation-based genome editing workflows.

Conventional approaches typically require transformation of plant cells, selection of transformed events, and regeneration of whole plants from tissue culture. These steps introduce significant time and resource requirements.

### Observations

Virus-based delivery enabled genome editing directly in intact plants and reduced reliance on tissue culture and regeneration. Systemic delivery allowed editing events to occur throughout the plant, including tissues relevant for heritable transmission.

These observations highlight the advantages of virus-based genome editing systems over conventional transformation-based approaches.

1. A method for producing a plant comprising a heritable genome modification without tissue culture or regeneration, the method comprising:
introducing into an intact plant at least one virus-based vector capable of systemic movement within the plant,
wherein the virus-based vector encodes
(a) a programmable site-specific nuclease or a fragment thereof that is structurally configured to satisfy viral cargo size and stability constraints while retaining genome editing activity, and
(b) at least one guide RNA or targeting sequence that directs the nuclease to a target locus in a genome of the plant,
wherein systemic viral movement results in genome editing in meristematic tissues, reproductive tissues, or tissues capable of giving rise to meristematic or germline tissues of the plant, thereby producing progeny plants comprising the heritable genome modification.

2. The method of claim 1, wherein the programmable site-specific nuclease is a compact nuclease encoded as a single open reading frame suitable for viral delivery.

3. The method of claim 2, wherein the compact nuclease is selected from compact CRISPR-associated nucleases, including but not limited to Cas12f, Cas121, CasΦ, or functional variants thereof.

4. The method of claim 1, wherein the programmable site-specific nuclease is expressed as a split nuclease comprising two or more inactive fragments that are reconstituted into a functional nuclease within plant cells.

5. The method of claim 4, wherein the split nuclease is derived from a CRISPR-associated nuclease, including but not limited to Cas9 or Cas12a.

6. The method of claim 4, wherein the split nuclease fragments are fused to complementary intein domains that mediate protein splicing to reconstitute the functional nuclease in planta.

7. The method of claim 1, wherein the programmable site-specific nuclease or fragment thereof is configured to comply with cargo size limitations imposed by the virus-based vector.

8. The method of claim 7, wherein the programmable site-specific nuclease or fragment thereof has a coding sequence length that does not exceed the cargo capacity of the virus-based vector.

9. The method of claim 8, wherein the cargo capacity of the virus-based vector for the programmable site-specific nuclease is no more than 2600 base pairs.

10. The method of claim 1, wherein the guide RNA or targeting sequence is encoded within a guide RNA cassette having a total length that does not exceed the cargo capacity of the virus-based vector.

11. The method of claim 10, wherein the guide RNA cassette has a total length of no more than 300 base pairs.

12. The method of claim 1, wherein the virus-based vector is a DNA virus or an RNA virus capable of systemic movement within the plant.

13. The method of claim 1, wherein the virus-based vector systemically delivers the programmable site-specific nuclease to vegetative tissues and tissues involved in reproductive development of the plant.

14. The method of claim 1, wherein the guide RNA is encoded by the same virus-based vector as the programmable site-specific nuclease or by a second virus-based vector capable of co-infecting the plant.

15. The method of claim 1, wherein the heritable genome modification comprises an insertion, deletion, substitution, or a combination thereof at the target locus.

16. The method of claim 1, wherein the heritable genome modification is detectable in seeds produced by the plant.

17. A virus-mediated genome editing system for producing heritable genome modifications in plants without regeneration, comprising:
at least one virus-based vector capable of systemic movement in a plant, and
a genome editing payload encoded by the virus-based vector, the genome editing payload comprising
(a) a programmable site-specific nuclease selected from compact nucleases or split nucleases, including but not limited to the nuclease types recited in claims 3 and 5, and
(b) at least one guide RNA or targeting sequence,
wherein the genome editing payload is configured to enable genome editing in meristematic tissues, reproductive tissues, or tissues capable of giving rise to meristematic or germline tissues of the plant.

18. A nucleic acid construct comprising a viral genome or a replication-competent portion thereof and a polynucleotide encoding a programmable site-specific nuclease or a fragment thereof,
wherein the construct is capable of replication and systemic movement in a plant and introduces a heritable genome modification when expressed in the plant.

19. A plant produced by the method of claim 1, wherein the plant comprises the heritable genome modification.

20. A progeny plant or seed derived from the plant of claim 19, wherein the progeny plant or seed comprises the heritable genome modification.
